(19) United States
(12) Patent Application Publication
(10) Pub. No.: US 2024/0033576 A1
(43) Pub. Date: Feb. 1, 2024

(54) EXERCISE COMMUNITY FORMATION SYSTEM, EXERCISE COMMUNITY FORMATION METHOD, AND COMPUTER READABLE MEDIUM

(71) Applicant: TOYOTA JIDOSHA KABUSHIKI KAISHA, Toyota-shi (JP)

(72) Inventors: Issei NAKASHIMA, Tokyo-to (JP); Toru MIYAGAWA, Seto-shi (JP); Hideo HASEGAWA, Nagoya-shi (JP); Yusuke KINOSHITA, Tokyo-to (JP); Makoto ASO, Mishima-shi (JP); Takatoshi MORIMITSU, Toyota-shi (JP); Aya ISHIHARA, Tokyo-to (JP); Hiroshi SHIMADA, Tajimi-shi (JP)

(73) Assignee: TOYOTA JIDOSHA KABUSHIKI KAISHA, Toyota-shi (JP)

(21) Appl. No.: 18/215,968

(22) Filed: Jun. 29, 2023

(30) Foreign Application Priority Data
Aug. 1, 2022 (JP) 2022-122663

(51) Int. Cl. A63B 24/00; A63B 71/06

(52) U.S. Cl. CPC A63B 24/0075; A63B 24/0062; A63B 71/0619; A63B 2024/0065; A63B 2024/0068

(57) ABSTRACT

An exercise community formation apparatus includes an exercise-related information acquisition unit configured to acquire exercise-related information for each of a plurality of members belonging to a specific community, an extraction unit configured to extract at least two or more members from the plurality of members based on the exercise-related information for each of the plurality of members, and an output unit configured to output a result of the extraction. The exercise-related information indicates a proficiency level for each of at least one or more exercise subjects of the corresponding member and a time period to which the corresponding member can assign an exercise.

### CROSS REFERENCE TO RELATED APPLICATIONS

This application is based upon and claims the benefit of priority from Japanese patent application No. 2022-122663, filed on Aug. 1, 2022, the disclosure of which is incorporated herein in its entirety by reference.

### BACKGROUND

The present disclosure relates to an exercise community formation system, an exercise community formation method, and a computer readable medium.

International Patent Publication No. WO 2019/146195 discloses an information processing apparatus that acquires sensor data obtained by sensing members belonging to a specific community, and automatically estimates life rhythms of the members belonging to the specific community based on the sensor data. A life rhythm refers to periodic life behaviors such as wake-up time, bedtime, meal time, work time, exercise time, bath time, toilet time, media viewing time, and family gathering time. The information processing apparatus presents results of estimating the life rhythms of the members belonging to the specific community. Based on the estimated life rhythms, the information processing apparatus searches for people in a community other than the specific community who fit certain conditions. For example, the information processing apparatus searches for people running during the same time period and presents the results of this search. This creates opportunities for people doing the same exercise during the same time period to perform the exercise together.

### SUMMARY

However, even if a plurality of members started to perform the same exercise subject together in the same time period, this performance does not always last for a long time.

An object of the present disclosure is to create a habit by which a plurality of members perform the same exercise subject together in the same time period, as well as to provide a technique for making the habit last.

According to a first aspect of the present disclosure, an exercise community formation system includes:
an exercise-related information acquisition unit configured to acquire exercise-related information for each of a plurality of members belonging to a specific community, the exercise-related information indicating a proficiency level for each of at least one or more exercise subjects of the corresponding member and a time period to which the corresponding member can assign an exercise;
an extraction unit configured to extract at least two or more members from the plurality of members based on the exercise-related information for each of the plurality of members; and
an output unit configured to output a result of the extraction is provided.

According to the above configuration, it is possible to create a habit by which a plurality of members perform the same exercise subject together in the same time period as well as to make the habit last.

The extraction unit may extract the at least two or more members so that
the at least two or more members can assign the exercise to the same time period, and
the at least two or more members have a common exercise subject such that a difference between proficiency levels of the at least two or more members satisfies a predetermined condition.

According to the above configuration, it is possible to prolong the habit by which a plurality of members perform the same exercise subject together in the same time period.

The exercise community formation system may further include:
a personality information acquisition unit configured to acquire personality information for each of the plurality of members, wherein
the extraction unit may extract the at least two or more members from the plurality of members based on the exercise-related information and the personality information for each of the plurality of members.

According to the above configuration, it is possible to prolong the habit by which a plurality of members perform the same exercise subject together in the same time period.

The exercise community formation system may further include a family structure information acquisition unit configured to acquire family structure information for each of the plurality of members.

The extraction unit may extract the at least two or more members from the plurality of members based on the exercise-related information and the family structure information for each of the plurality of members.

According to the above configuration, it is possible to prolong the habit by which a plurality of members perform the same exercise subject together in the same time period.

The exercise-related information may further indicate a purpose of the corresponding member for performing the exercise.

According to the above configuration, it is possible to prolong the habit by which a plurality of members perform the same exercise subject together in the same time period.

The exercise-related information may further indicate an acceptable exercise load of the corresponding member.

According to the above configuration, it is possible to prolong the habit by which a plurality of members perform the same exercise subject together in the same time period.

The proficiency level may correspond to years of experience in the corresponding exercise subject.

According to the above configuration, the proficiency level is objectively determined.

The proficiency level may correspond to whether or not a member is proficient in a specific action in the corresponding exercise subject.

According to the above configuration, the proficiency level is objectively determined.

According to a second aspect of the present disclosure, an exercise community formation method performed by a computer includes:
acquiring exercise-related information for each of a plurality of members belonging to a specific community, the exercise-related information indicating a proficiency level for each of at least one or more exercise subjects of the corresponding member and a time period to which the corresponding member can assign an exercise;
extracting at least two or more members from the plurality of members based on the exercise-related information for each of the plurality of members; and
outputting a result of the extraction is provided.

According to the above method, it is possible to create a habit of a plurality of members performing the same exercise subject together in the same time period as well as to make the habit last.

A program for causing a computer to execute the exercise community formation method is provided.

According to the present disclosure, it is possible to create a habit by which a plurality of members perform the same exercise subject together in the same time period as well as to make the habit last.

The above and other objects, features and advantages of the present disclosure will become more fully understood from the detailed description given hereinbelow and the accompanying drawings which are given by way of illustration only, and thus are not to be considered as limiting the present disclosure.

system;

### BRIEF DESCRIPTION OF DRAWINGS

FIG. 1 is an overview diagram showing a configuration of a community

FIG. 2 is a functional block diagram of an exercise community formation apparatus;

FIG. 3 is an explanatory diagram of member information; and

FIG. 4 is a control flow of the exercise community formation apparatus.

### DESCRIPTION OF EMBODIMENTS

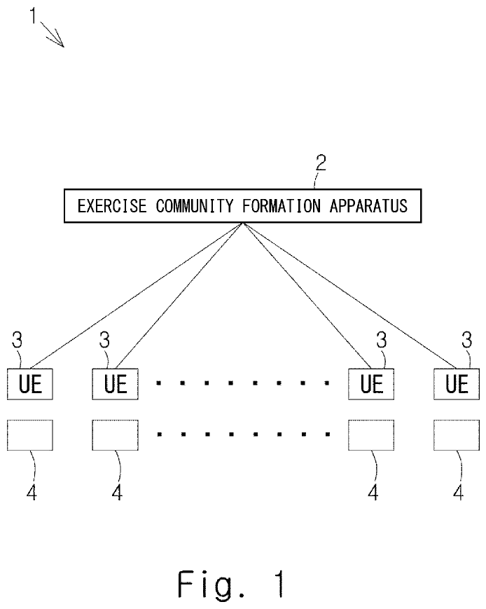

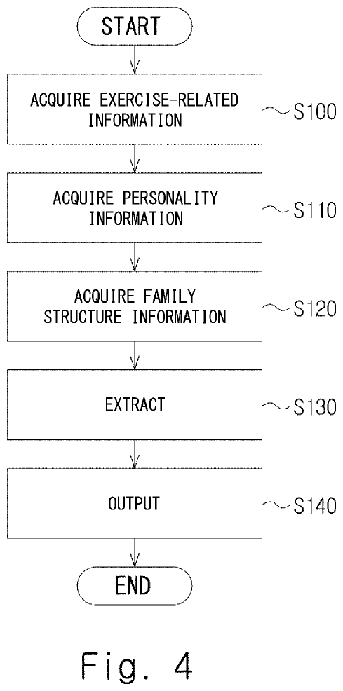

Embodiments of the present disclosure are described below with reference to FIGS. 1 to 4.

FIG. 1 is an overview diagram showing a configuration of a community system 1. As shown in FIG. 1, the community system 1 includes an exercise community formation apparatus 2 and a plurality of UEs 3. The plurality of UEs 3 are configured to be bidirectionally communicable with the exercise community formation apparatus 2, typically via an Internet line. Each of the plurality of UEs 3 is an information processing terminal owned by each of a plurality of members 4. The plurality of members 4 belong to a specific community. Belonging to a specific community means, for example, living in the same area, working for the same company, or using the same SNS service.

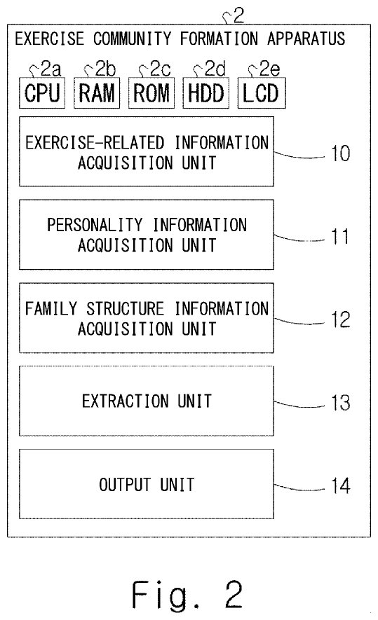

FIG. 2 is a functional block diagram of the exercise community formation apparatus 2. As shown in FIG. 2, the exercise community formation apparatus 2 includes a CPU 2a (Central Processing Unit), a RAM 2b (Random Access Memory) that can be freely read and written, and a ROM 2c (Read Only Memory). The exercise community formation apparatus 2 further includes an HDD 2d (Hard Disk Drive) and an LCD 2e (Liquid Crystal Display). The CPU 2a reads and executes a control program stored in the ROM 2c, and thus the control program causes hardware such as the CPU 2a to function as an exercise-related information acquisition unit 10, a personality information acquisition unit 11, a family structure information acquisition unit 12, an extraction unit 13, and an output unit 14.

The exercise-related information acquisition unit 10 acquires exercise-related information for each of the plurality of members 4. Specifically, each member 4 inputs his/her exercise-related information to the UE 3. The exercise-related information acquisition unit 10 acquires the exercise-related information from the UEs 3. The exercise-related information acquisition unit 10 stores the acquired exercise-related information in the HDD 2d.

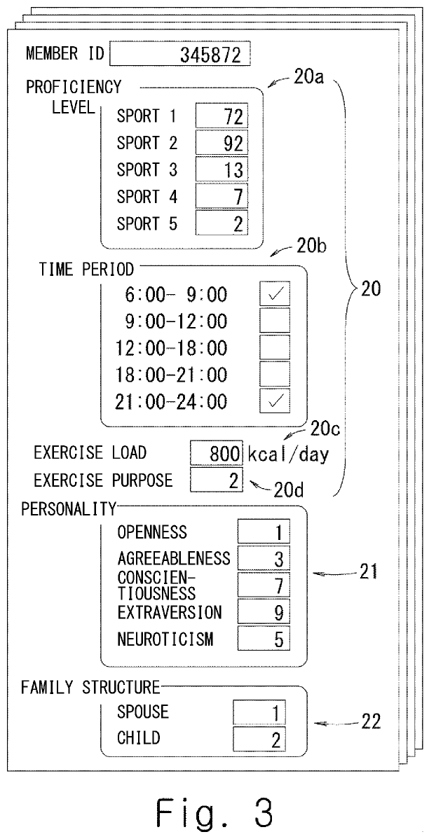

FIG. 3 shows member information about each member 4 stored in the HDD 2d. The member information includes exercise-related information 20, personality information 21, and family structure information 22.

As shown in FIG. 3, the exercise-related information 20 shows a proficiency level 20a for each exercise subject of the corresponding member 4, time periods 20b to which the corresponding member 4 can assign exercises, an acceptable exercise load 20c of the corresponding member 4, and a purpose 20d for performing exercises of the corresponding member 4.

“Sport 1” to “Sport 5” shown in FIG. 3 can be easily undertaken in the member 4′s spare time and are typically tennis, golf, running, cycling, and weight training (workout).

The proficiency level for each exercise subject can typically be expressed as a number from 0 to 100. In this embodiment, the higher the number indicating the proficiency level 20a, the higher the proficiency level.

The proficiency level for each exercise subject may be directly input by the member 4 on the UE 3.

Alternatively, the exercise-related information acquisition unit 10 may determine the proficiency level for each exercise subject according to years of experience input by the member 4 on the UE 3 for each exercise subject. For example, if the member 4 inputs “I have been playing tennis for 15 years” on the UE 3, the exercise-related information acquisition unit 10 may determine the proficiency level in tennis to be 60 or higher. Also, if the member 4 inputs “I have just started running last month” on the UE 3, the exercise-related information acquisition unit 10 may determine the proficiency level in running to be 5 or lower.

In addition, the proficiency level of the exercise-related information acquisition unit 10 may be determined for each exercise subject according to whether or not the member 4 has acquired a specific action input on the UE 3. For example, when the member 4 inputs “I can hit a topspin serve in tennis” on the UE 3, the exercise-related information acquisition unit 10 may determine the proficiency level in tennis to 70 or higher. Also, when the member 4 inputs “I can finish a full marathon” on the UE 3, the exercise-related information acquisition unit 10 may determine the proficiency level in running to be 80 or higher.

The member 4 shown as an example in FIG. 3 has a proficiency level of 72 in Sport 1, a proficiency level of 92 in Sport 2, and low proficiency levels in all other exercise subjects.

The time periods that the member 4 shown as an example in FIG. 3 can assign exercises are “6:00-9:00” and “21:00 to 24:00”.

The acceptable exercise load for the member 4 shown as an example in FIG. 3 is 800 kcal/day. The acceptable exercise load for the member 4 represents the acceptable exercise load for the member 4 in one exercise.

The purpose of exercising typically falls into five categories. The five categories are typically: “stress reduction”, “diet”, “health promotion”, “muscle building”, and “time killer”. The purpose of the member 4, who is shown as an example in FIG. 3, to exercise is “diet (Category 2)”.

The personality information acquisition unit 11 acquires the personality information 21 for each of the plurality of members 4. Specifically, each member 4 inputs his/her personality information 21 to the UE 3. The personality information acquisition unit 11 acquires the personality information 21 from the UEs 3. The personality information acquisition unit 11 stores the acquired personality information in the HDD 2d.

The personality information is typically Big Five information and composed of five characteristic values: “openness”, “agreeableness”, “conscientiousness”, “extraversion”, and “neuroticism”. The characteristic values can typically be expressed as a number from 0 to 10. The member 4, shown as an example in FIG. 3, is extroverted and sincere but lacks openness and agreeableness.

The family structure information acquisition unit 12 acquires the family structure information 22 for each of the plurality of members 4. Specifically, each member 4 inputs his/her family structure information 22 to the UE 3. The family structure information acquisition unit 12 acquires the family structure information 22 from the UEs 3. The family structure information acquisition unit 12 stores the acquired family structure information in the HDD 2d.

The family structure information 22 typically indicates the presence or absence of a spouse and the number of children. The member 4, shown as an example in FIG. 3, has a spouse and two children.

The extraction unit 13 extracts at least two or more members 4 from the plurality of members 4 based on the exercise-related information 20, the personality information 21, and the family structure information 22 about each of the plurality of members 4.

### (Extraction Based on Exercise-Related Information 20)

The extraction unit 13 extracts at least two or more members so that the at least two or more members can assign an exercise to the same time period, and the at least two or more members have a common exercise subject such that a difference between proficiency levels of the at least two or more members satisfies a predetermined condition. Here, the “difference between the proficiency levels satisfy a predetermined condition” typically means, but is not limited to, that the difference between the proficiency levels is less than a first threshold. The first threshold is typically, but is not limited to, 5, 10, or 15, but the present disclosure is not limited to this.

That is, for example, in the case of tennis, a difference between the proficiency level of a member 4 who only started playing tennis last month and the proficiency level of a member 4 who has played in a prefectural tennis tournament would be so great that it would be virtually difficult for them to enjoy tennis together. Instead, a member 4, who has five years of tennis experience, and a member 4, who also has seven years of tennis experience, will enjoy tennis together, because there is not much difference in their proficiency levels of tennis.

Therefore, if the extraction unit 13 extracts at least two or more members as described above, the extracted plurality of members 4 will be able to enjoy the same exercise subjects together over a long period of time in their spare time.

The extraction unit 13 may further narrow down the extracted members 4 so that at least two or more members 4 have the same purpose of performing their exercises. As a result, a relationship of encouraging each other to achieve the same goal develops among the extracted members 4, so that they will be able to enjoy the same exercise subjects together for a long period of time in their spare time.

The extraction unit 13 may further narrow down the extracted members 4 so that the difference between the acceptable exercise loads of the at least two or more members 4 becomes less than a predetermined value. This allows the extracted members 4 to exercise with an exercise load that is comfortable to each of them, so that they will enjoy the same exercise subject together for a longer period of time in their spare time.

### (Extraction Based on Personality Information 21)

The extraction unit 13 extracts at least two or more members whose personalities are similar each other. Specifically, when all the differences between the five characteristic values of a member A and those of a member B are 3 or less, the extraction unit 13 determines that the personalities of the member A and the member B are similar to each other and extracts the member A and the member B from the plurality of members 4. The member A and the member B will be able to enjoy the same exercise subject together for a long period of time in their spare time, because their personalities are similar.

### (Extraction Based on Family Structure Information 22)

The extraction unit 13 extracts at least two or more members whose family structures are similar to each other. Specifically, when a family structure of a member C in which a spouse is present or absent and a child is present or absent is the same as that of a member D, the extraction unit 13 determines that the family structure of the member C is similar to that of the member D and extracts the members C and D from the plurality of members 4. Since the members C and D have similar family structures, family relationships will be successful.

Next, the extraction unit 13 extracts at least two or more members 4 from the plurality of members 4 based on the extraction result based on the exercise-related information 20, the extraction result based on the personality information 21, and the extraction result based on the family structure information 22. In this embodiment, the extraction unit 13 emphasizes the extraction result based on the exercise-related information 20, and by correcting the extraction result based on the exercise-related information 20 with the extraction result based on the personality information 21 and the extraction result based on the family structure information 22, the extraction unit 13 extracts at least two or more members 4 from the plurality of members 4.

For example, if the extraction result based on the exercise-related information 20 is members E, F, and G, the extraction result based on the personality information 21 is members E, F, G, and H, and the extraction result based on the family structure information 22 is members F, G, and H, the extraction unit 13 may extract the members F and G, who are at least included in the extraction result based on the exercise-related information 20 and also included in the extraction result based on the personality information 21 and the extraction result based on the family structure information 22.

The output unit 14 outputs the result of the extraction obtained by the extraction unit 13. Specifically, the output unit 14 outputs the result of the extraction to the UEs 3 of the plurality of members 4 extracted by the extraction unit 13. Alternatively, the output unit 14 may output the result of the extraction to the LCD 2e. When the output unit 14 outputs the result of the extraction to the LCD 2e, the plurality of members 4 can check who has been extracted by checking the LCD 2e.

The output unit 14 may simultaneously output member IDs of the plurality of members 4 extracted by the extraction unit 13. The result of the extraction may simultaneously show the time period common to the plurality of members 4 extracted by the extraction unit 13 and to which exercise can be assigned. The result of the extraction may simultaneously show the exercise subject that the plurality of members 4 extracted by the extraction unit 13 are proficient at about the same level.

Next, the operation of the exercise community formation apparatus 2 is described.

### S100:

First, the exercise-related information acquisition unit 10 acquires the exercise-related information for each of the plurality of members 4.

### S110:

Next, the personality information acquisition unit 11 acquires the personality information for each of the plurality of members 4.

### S120:

Next, the family structure information acquisition unit 12 acquires the family structure information for each of the plurality of members 4.

### S130:

Next, the extraction unit 13 extracts at least two or more members from the plurality of members 4 based on the exercise-related information, the personality information, and the family structure information for each of the plurality of members 4.

### S140:

Next, the output unit 14 outputs the result of the extraction obtained by the extraction unit 13.

The extracted members 4 will then communicate with each other to perform the same exercise subject together in the same time period. At this time, the proficiency levels of the members 4 will be similar to each other, so that they will be able to enjoy this exercise subject without any problems, thereby prolonging the habit of exercising together.

Embodiments of the present disclosure have been described above. The above embodiment has the following features.

The exercise community formation apparatus 2 (exercise community formation system) includes the exercise-related information acquisition unit 10 configured to acquire the exercise-related information for each of the plurality of members 4 belonging to a specific community, the extraction unit 13 configured to extract at least two or more members 4 from the plurality of members 4 based on the exercise-related information for each of the plurality of members 4, and the output unit 14 configured to output a result of the extraction. The exercise-related information indicates a proficiency level for each of at least one or more exercise subjects of the corresponding member 4 and a time period to which the corresponding member 4 can assign an exercise. According to the above configuration, it is possible to create a habit by which a plurality of members 4 perform the same exercise subject together in the same time period as well as to make the habit last.

In the above embodiments, the exercise-related information indicates the proficiency level for each of the five exercise subjects of the corresponding member 4. Alternatively, the exercise-related information may indicate the proficiency level for each of one or more exercise subjects of the corresponding member 4. That is, the exercise-related information may indicate the proficiency level for each of at least one or more exercise subjects of the corresponding member 4.

Furthermore, the extraction unit 13 extracts the at least two or more members so that the at least two or more members 4 can assign the exercise to the same time period, and the at least two or more members 4 have a common exercise subject such that a difference between proficiency levels of the at least two or more members 4 satisfies a predetermined condition. According to the above configuration, it is possible to prolong the habit by which the plurality of members 4 perform the same exercise subject together in the same time period.

The exercise community formation apparatus 2 further includes the personality information acquisition unit 11 configured to acquire personality information for each of the plurality of members 4. The extraction unit 13 extracts the at least two or more members 4 from the plurality of members based on the exercise-related information and the personality information for each of the plurality of members 4. According to the above configuration, a low-stress relationship is achieved, so that the habit by which the plurality of members 4 perform the same exercise subject together in the same time period can be effectively prolonged.

The exercise community formation apparatus 2 further includes a family structure information acquisition unit 12 configured to acquire family structure information for each of the plurality of members 4. The extraction unit 13 may extract at least two or more members from the plurality of members 4 based on the exercise-related information and family structure information for each of the plurality of members 4. According to the above configuration, the development of a family-wide relationship can effectively prolong the habit by which the plurality of members perform the same exercise subject together in the same time period.

The exercise-related information may further indicate a purpose of the corresponding member for performing the exercise. According to the above configuration, a relationship of encouraging each other to achieve the same goal develops among the extracted members 4, so that they will be able to enjoy the same exercise subjects together for a long period of time in their spare time.

The exercise-related information may further indicate the acceptable exercise load of the corresponding member. This allows the extracted members 4 to exercise with an exercise load that is comfortable to each of them, so that they will enjoy the same exercise subject together for a longer period of time in their spare time.

The proficiency level may correspond to years of experience in the corresponding exercise subject. According to the above configuration, the proficiency level is objectively determined.

The proficiency level may correspond to whether or not a member is proficient in a specific action in the corresponding exercise subject. According to the above configuration, the proficiency level is objectively determined. The exercise-related information acquisition unit 10 may be acquired from a storage medium such as a CDROM or DVDROM instead of acquiring the exercise-related information, the personality information, and the family structure information from the UE 3 owned by each member 4.

The exercise community formation apparatus 2 may be implemented by a single computer or by distributed processing by plurality of computers.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. An exercise community formation system comprising:
at least one memory storing instructions and
at least one processor configured to execute the instructions to;
acquire exercise-related information for each of a plurality of members belonging to a specific community, the exercise-related information indicating a proficiency level for each of at least one or more exercise subjects of the corresponding member and a time period to which the corresponding member can assign an exercise;
extract at least two or more members from the plurality of members based on the exercise-related information for each of the plurality of members; and
output a result of the extraction.

2. The exercise community formation system according to claim 1, wherein the at least one processor is further configured to execute the instructions to:
extract the at least two or more members so that
the at least two or more members can assign the exercise to the same time period, and
the at least two or more members have a common exercise subject such that a difference between proficiency levels of the at least two or more members satisfies a predetermined condition.

3. The exercise community formation system according to claim 1, wherein the at least one processor is further configured to execute the instructions to:
acquire personality information for each of the plurality of members, and
extract the at least two or more members from the plurality of members based on the exercise-related information and the personality information for each of the plurality of members.

4. The exercise community formation system according to claim 1, wherein the at least one processor is further configured to execute the instructions to:
acquire family structure information for each of the plurality of members, and
extract the at least two or more members from the plurality of members based on the exercise-related information and the family structure information for each of the plurality of members.

5. The exercise community formation system according to claim 1, wherein
the exercise-related information further indicates a purpose of the corresponding member for performing the exercise.

6. The exercise community formation system according to claim 1, wherein
the exercise-related information further indicates an acceptable exercise load of the corresponding member.

7. The exercise community formation system according to claim 1, wherein
the proficiency level corresponds to years of experience in the corresponding exercise subject.

8. The exercise community formation system according to claim 1, wherein
the proficiency level indicates whether or not the corresponding member is proficient in a specific action in the corresponding exercise subject.

9. A computer-implemented exercise community formation method comprising:
acquiring exercise-related information for each of a plurality of members belonging to a specific community, the exercise-related information indicating a proficiency level for each of at least one or more exercise subjects of the corresponding member and a time period to which the corresponding member can assign an exercise;
extracting at least two or more members from the plurality of members based on the exercise-related information for each of the plurality of members; and
outputting a result of the extraction.

10. A non-transitory computer readable medium storing a program for causing a computer to execute the computer-implemented exercise community formation method according to claim 9.
